(19) United States
(12) Patent Application Publication
(10) Pub. No.: US 2003/0083588 A1
(43) Pub. Date: May 1, 2003

(54) Pre-screening utilizing magnetic resonance imaging field

(76) Inventors: Richard J. McClure, San Diego, CA (US); R. Kemp Massengill, Leucadia, CA (US); William F. Avrin, San Diego, CA (US)

Correspondence Address: GERALD W SPINKS, P. O. BOX 2330, PORT ORCHARD, WA, 98366, US

(21) Appl. No.: 10/244,109

(22) Filed: Sep. 12, 2002

Related U.S. Application Data
(63) Continuation-in-part of application No. 10/017,913, filed on Oct. 29, 2001.
(60) Provisional application No. 60/366,799, filed on Mar. 22, 2002.
(60) Provisional application No. 60/385,056, filed on May 31, 2002.

(51) Int. Cl. A61B 5/05

(52) U.S. Cl. 600/547

(57) ABSTRACT

A method for pre-screening a patient for the presence of a ferromagnetic foreign body, prior to performance of an MRI procedure, using a low power portion of the MRI field to magnetize the foreign body for detection purposes.

### CROSS REFERENCE TO RELATED APPLICATIONS

This is a continuation-in-part patent application of co-pending U.S. patent application Ser. No. 10/017,913, filed on Oct. 29, 2001, and entitled “Ferromagnetic Foreign Body Detection Utilizing Eye Movement”. This application also claims priority from U.S. Provisional Patent Application Serial No. 60/366,799, filed on Mar. 22, 2002, and entitled “Pre-Screening Utilizing Magnetic Resonance Imaging Field”, and from U.S. Provisional Patent Application Serial No. 60/385,056, filed on May 31, 2002, and entitled “Pre-Screening Utilizing Magnetic Resonance Imaging Field”.

### STATEMENT REGARDING FEDERALLY SPONSORED RESEARCH OR DEVELOPMENT

Not Applicable

### BACKGROUND OF THE INVENTION

1. Field of the Invention

The present invention is in the field of human magnetic resonance imaging.

2. Background Art

Patients requiring magnetic resonance imaging (MRI) studies will obligatorily be exposed to very large magnetic fields. These magnetic fields are of sufficient size to cause motion of magnetizable particles in the body.

The body area most susceptible to damage is the eye, because of the general absence within the eye of constraining structures. Severe intraocular damage has been documented in MRI procedures, including irreversible loss of vision. Another area where severe damage can occur is the brain, and ferromagnetic particles, such as clips from neurosurgical procedures, can move when the large field associated with magnetic resonance imaging is applied, thereby wreaking havoc within brain tissue. Although neurosurgical clips in present use are generally manufactured from non-magnetic materials, in the past, clips were typically ferromagnetic in nature.

The most devastating effect of a ferromagnetic foreign body (FFB) within the eye, the orbit, or the brain is the torque effect induced by the MRI magnetic field upon the ferromagnetic object. Objects which are spherical do not rotate in the MRI magnetic field; however, most objects are not spherical. Therefore, most objects will tend to align with the magnetic field. For an elongated ferromagnetic foreign body, this alignment is akin to a “propeller” effect, which can result in cutting through ocular or neurological tissues.

In most areas of the body, although damage can occur, the damage tends to be less extensive, because the body forms scar tissues and essentially encapsulates the ferromagnetic foreign body. This encapsulation does not occur to any significant degree within the eye, the orbit, or the brain. The main areas of risk for MRI scanning, then, are the eye, the orbit, and the brain. The eye can have retained ferromagnetic foreign bodies which are subjected to forces or torque which pull or rotate the object through the ocular tissues, which may cause a permanent loss of vision. Obviously, welders and machinists are at particularly high risk. The clinical literature contains case studies of patients with previously unknown intraocular ferromagnetic foreign bodies suffering blindness during or after an MRI scan. Motion of the FFB can cause deleterious effects, such as vitreous hemorrhage, retinal damage, or damage to the optic nerve. A similar problem exists within the brain, since a retained intracranial ferromagnetic foreign body can be moved through brain tissue under the influence of the large magnetic field of the MRI instrument. Tragic neurological deficit may be a consequence.

The significance of the problems encountered is related to the large size of the magnetic field during magnetic resonance imaging. A typical MRI system generates a magnetic field strength between 10,000 Oersted (Oe) and 15,000 Oe, or 1 to 1.5 Tesla (T). The MRI field cannot be readily switched off, so the patient is inserted into the MRI machine with the magnetic field activated. During positioning of the patient within the MRI magnetic field, the patient and, therefore, a retained FFB, experience an increasing magnetic field, which may exert torque on the FFB, causing a rotational movement of the object, especially in the eye, the orbit, or the brain, and, in high magnetic field gradient regions, also a translational force, tending to move the particle if it is not encapsulated by scar tissue. A particularly acute potential problem exists in the eye, the orbit, or the brain, where tissues constraining movement are minimal. The magnetic field of the MRI system is very large, extending for a distance of several feet. Thus, for example, when the knee is scanned, a large magnetic field is applied not only to the knee, but also to the eye, to the orbit, and to the brain. Therefore, even during magnetic resonance imaging of the knee, torquing and/or translational movement of a retained FFB within the eye, orbit, or brain, can occur.

Damage can arise not only in conventional MRI units with magnetic field strengths of 1 Tesla or higher, but has also been documented in an MRI instrument with a field strength of 0.35 Tesla (3,500 Oe). Laboratory studies with freshly harvested animal and cadaver eyes, however, have found that intraocular and intraorbital FFB's show the most significant movement during an MRI procedure with larger fields, such as 1 to 1.5 Tesla. In the absence of a suitable and practical pre-MRI screening test, the consensus in the literature is that the risk of trauma from retained FFB's for metal workers is often sufficient to withhold magnetic resonance imaging, at least at magnetic field strengths greater than 0.35 Tesla.

The known presence of ferromagnetic foreign bodies in the eye, orbit, or brain, and sometimes elsewhere, is currently an established contraindication for MRI procedures. However, a patient often does not know when a foreign object is present, or the patient does not provide such information to the radiologist or technician. Therefore, radiologists performing MRI scanning may be unaware that a patient has a retained ferromagnetic foreign body in a critical body region, with potentially catastrophic consequences.

Risk determination associated with medical procedures is often difficult. Physicians must daily weigh the costs and risks of diagnostic procedures against the information gained, sometimes with inadequate information as to the probability of an adverse reaction. In the case of MRI, the availability of a low-cost, low-risk screening procedure to detect FFB's would allow physicians to make the correct decision in an informed way, thereby avoiding the risk of damage without depriving patients needlessly of a vital diagnostic modality.

X-ray radiography has shown mixed results in detecting foreign bodies within or near the eye. Although the technology provides generally excellent resolution of even tiny FFB's, the fact that an x-ray must be interpreted can lead to human error. CT procedures have certain advantages, but CT scans are too expensive for routine screening purposes. Both CT scans and X-ray radiography share another serious problem, in that these modalities can differentiate between metallic and nonmetallic foreign bodies, but not between ferromagnetic and non-ferromagnetic foreign bodies. If all patients having retained metallic objects, some of which were ferromagnetic, and some of which are not ferromagnetic, were denied the application of MRI scanning, a significant number of these patients would be needlessly deprived of a valuable diagnostic procedure. To base a clinical decision to perform, or not to perform, an MRI procedure upon plain X-ray films showing a metallic foreign body is undesirable, as the X-ray does not answer vital questions regarding the ferromagnetic nature of the retained metallic foreign object in question. This problem, together with the fact that ionizing radiation is inherent in radiological tests, and, additionally, the occasional failure because of human interpretational error to detect significant and potentially dangerous objects, are drawbacks inherent with X-ray and CT techniques for routine pre-MRI screening.

Ultrasound is another methodology which can be employed for detecting foreign bodies in or near the eye, or elsewhere. One study documented that ultrasound has a probability of detection, for all types of foreign body materials, of 93%. Ultrasound procedures suffer the same deficiency as X-ray and CT procedures, however, in that ultrasound cannot distinguish ferromagnetic from non-ferromagnetic foreign bodies. Passive magnetic detection, as used in magnetoencephalography (MEG), can detect the perturbation of the earth's magnetic field caused by a ferromagnetic object. Algorithms exist which allow localization and characterization of an object in question by measuring a complete set of magnetic field and gradient tensor components. However, the ultra-sensitive magnetometers used for MEG are exceedingly expensive. Furthermore, the static field perturbations caused by an FFB can be difficult to distinguish from background fields.

It would be advantageous, then, to have a practical ferromagnetic foreign body detection system designed to screen patients before the commencement of magnetic resonance imaging. With the tremendously increasing popularity of magnetic resonance imaging, such screening could reduce the morbidity and human suffering caused by MRI-induced movement of ferromagnetic foreign bodies within, or adjacent to, vital organs.

It is an object of the present invention to provide a method and apparatus for the detection of ferromagnetic foreign bodies retained within a region of a patient before that patient undergoes magnetic resonance imaging. It is a further object of the present invention to provide a method and apparatus for determining in advance of the MRI procedure itself the location and size of a retained ferromagnetic foreign body within the diagnostic region of interest of a patient. Finally, it is an object of the present invention to provide a noninvasive, safe, economical, and easy-to-use method and apparatus for detection, location, and characterization of a retained ferromagnetic foreign body.

### BRIEF SUMMARY OF THE INVENTION

Briefly stated, the present invention includes a method for detecting ferromagnetic foreign bodies in a sensed region of the body, such as the eye, the orbit, or the brain, wherein the foreign body is magnetized for detection by the fringing magnetic field of an MRI system and detected by the sensor system apparatus of the present invention. The present invention also includes an apparatus for detecting ferromagnetic foreign bodies in a sensed region of the body, such as the eye, the orbit, or the brain, wherein the foreign body is magnetized for detection by the fringing magnetic field of an MRI system and detected by the sensor system of the present invention.

The present invention provides for the use of a portion of the magnetic resonance imaging (MRI) magnetic field to magnetize a retained ferromagnetic foreign body (FFB) in the eye, the orbit, the brain, or other selected body area, so that it can be detected by suitable methodology and instrumentation as described herein.

Prior to the present invention, the MRI procedure itself has been investigated for its ability to provide images of intraorbital and intraocular foreign bodies. However, the MRI procedure itself is unsuitable, and even dangerous, as a screening procedure except at very low magnetic field levels. At such low fields, however, the sensitivity of the MRI procedure is insufficient to detect many objects. Since most MRI systems run at fixed, large magnetic fields, low-field screening would required a separate MRI instrument. The cost of even an ultralow-field MRI instrument would be unacceptably high for routine screening, thus rendering this potential solution impractical.

In the present invention, the pre-MRI screening measurement, i.e., prior to the MRI procedure itself, is made in the fringing field portion of the MRI instrument's magnetic field, where the amplitude of interaction between the FFB particle and the MRI magnetic field is significantly less than that which occurs once the patient enters the operative MRI magnetic field within the MRI-measurement zone.

The preferred embodiment of the present invention utilizes the far-field on-axis (axial) portion of the fringing field of the MRI magnetic field. To define terms, the fringing field means the magnetic field of the MRI instrument external to the MRI-measurement zone itself. The far-field portion of the fringing field is where the magnetic field of the MRI magnet or magnets is significantly reduced. The far-field portion of the fringing field is preferred, because, in this region, the magnetic field which induces magnetization of the ferromagnetic foreign body is much reduced and, consequently, the forces exerted upon a ferromagnetic foreign body, if such is present, are very small and quite safe. Additionally, in the far-field portion of the MRI fringing field, the field gradient is small, and, therefore, the risk of causing translational motion of the FFB is minimal.

The preferred embodiment, then, uses the axial, that is, the on-axis, portion of the far-field fringing field. It is advantageous to utilize the on-axis portion for symmetry reasons, thereby allowing more readily for sensor nulling. Alternatively, off-axis regions of the far-field fringing field can be employed, but, in this alternate non-preferred embodiment, it is considerably more difficult to null the sensor because of the asymmetry of the off-axis field components.

The present invention utilizes one or more pre-MRI measurement zones, or “test zone set points,” which lie along the far-field axial portion of the MRI fringing field. At each test zone set point, beginning with that which corresponds to the lowest designated magnetic field strength, and proceeding in serial fashion to increasingly greater magnetic field strength test zone set points, the desired body area of the patient is screened for the presence of a ferromagnetic foreign body, as described in detail herein.

An alternate embodiment of the present invention utilizes a solitary test zone set point on the far-field portion of the MRI axial fringing magnetic field corresponding to a predetermined magnetic field strength, such as 100 Oe. This test zone set point represents 150 times less magnetic field strength than that present within the MRI-measurement zone of a 15,000 Oe (1.5 Tesla) MRI instrument. Thus, the relative risk of this embodiment's pre-MRI screening instrument is very small, indeed, when compared with that of the MRI procedure itself, as the risk of dislodging a FFB with a magnetic field strength of only 100 Oe, or even 200 Oe, is extremely minimal. With this alternate embodiment, under certain circumstances, a ferromagnetic foreign body which is in and of itself a magnet or a magnet fragment may escape detection. In such cases, an instrument with two or more test zone set points improves the chance for FFB detection.

The present invention requires a sensor. In the preferred embodiment, a 4-arm sensor bridge arrangement is used, which allows for temperature compensation. In order to read the output of the sensor bridge, a null is established, which means that the output, i.e., the signal voltage, from the bridge will be adjusted to read at zero. When a signal is detected due to the presence of a FFB, the bridge output voltage will deviate from zero, and this deviation is an indication of the presence of a FFB.

An alternative approach for pre-MRI screening of a desired body location, such as the eye, is to provide FFB detection by moving the sensor system transversely with respect to the fringing axial magnetic field of the MRI, or to move the sensor system in other areas of the non-axial fringing magnetic field of the MRI.

These approaches increase the complexity of the procedure, because of nulling challenges related to the changing off-axis, and therefore asymmetrical, magnetic field strengths emanating from the MRI instrument. Software can be written to compensate for these nulling problems; however, the complexity of these alternate embodiments can result in less reliability than employing the axial portion of the MRI fringing magnetic field.

Movement of the sensor system in any direction other than along the axial magnetic field of the MRI instrument will require rebalancing of the null, which is a more difficult problem, in that the null is continuously changing because of movement of the sensor system in an asymmetrical region of the fringing magnetic field of the MRI. These null problems are even more complicated with the 4-arm sensor system. For these reasons, use of the on-axis fringing magnetic field of the MRI instrument will normally be preferred. This latter approach has more inherent reliability and, therefore, more accurate measuring capability than the alternate embodiments.

Moving the sensor system to and fro along the axial portion of the fringing field of the MRI avoids the nulling problems inherent whenever non-axial portions of the fringing field are employed.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIG. 1 is a longitudinal section view of the magnetic field of a magnetic resonance imaging instrument;

FIG. 2 is a diagram of the diminution in magnetic field strength as the distance from the MRI measurement zone increases;

FIG. 3 is a schematic of the apparatus of the present invention in the magnetic field shown in FIG. 1;

FIGS. 4 through 6 are schematics showing the apparatus of the present invention at various test zone set points;

FIGS. 7 and 8 are elevation views of a patient positioned relative to the apparatus of the present invention;

FIGS. 9 through 11 show a patient positioned relative to the apparatus of the present invention, at the test zone set points shown in FIGS. 4 through 6;

FIGS. 12A and 12B show enlarged views of portions of the patient and the apparatus of the present invention; and

FIG. 13 is a section view of the magnetic field shown in FIG. 1, illustrating the placement of an ante-chamber.

### DETAILED DESCRIPTION OF THE INVENTION

An MRI apparatus provides a relatively large volume of magnetic field which extends into space external to the MRI-measurement zone and which diminishes in strength as the distance from the MRI-measurement zone increases. It is in the region external to the MRI-measurement zone, called the fringing field, that the MRI instrument's magnetic field utilized in the present invention decreases to a useful and safe value which is orders of magnitude lower than that encountered within the MRI-measurement zone itself. For instance, the 100 Oe magnetic field region of the MRI fringing field is 150 times less powerful than the magnetic field within the MRI-measurement zone of a 1.5 Tesla (15,000 Oe) MRI instrument. The distance from the MRI-measurement zone to the 100 Oe magnetic field region depends upon the model of MRI instrument being used. One Marconi machine has a 100 Oe distance of about one meter, while the 100 Oe distance in some of the older instruments may be several meters, or more.

In practice, the present MRI pre-screening invention measures designated locations, called “test zone set points,” of the magnetic fringing field emanating from MRI instrument itself, and, utilizing these measurements, the patient is advanced to a predetermined marked location at a distance from the MRI-measurement zone, such as that location which provides a magnetic field strength of 100 Oe. Other designated test zone set points can be employed, as desired and as described herein.

Whatever magnetic field strength of the MRI fringing field is used, the purpose of this magnetic field is to magnetize a ferromagnetic foreign body (if present), thereby allowing FFB detection by the sensor apparatus system of the present invention. The present invention, in essence, provides a DC magnetization measurement system in which the applied magnetic field is the fringing field of the MRI magnet or magnets.

The present invention comprises a sensor system into which the patient's sensed area, such as the eye, is placed within each designated magnetic field strength region, or test zone set point. The sensing system preferably employs magnetoresistive elements, although alternative sensor systems can be employed. Movement of the sensed region, such as of the eye, or the head, is employed to allow detection of the FFB-induced magnetization field, and furthermore, if desired, a bag of water, gel, or other suitable material, may be positioned between the sensor system and the body region to be sensed, to facilitate the pre-MRI screening measurements and to make the patient more comfortable. For pre-MRI screening of the eye, a moving eye-sensed target is preferably provided to encourage predictable and comfortable movement of the eye for signal detection considerations, i.e., to differentiate the FFB's coherent signal from background noise. In an instrument configuration of the present invention utilized to screen the orbit and the brain, head movement is used to facilitate sensing an FFB within the brain (such as a neurological clip previously implanted during brain surgery), or within the orbit.

The size of the magnetic field which is considered useful depends upon the nature of the FFB to be sensed. Test zone set points can be selected in regions in which the MRI fringing field is larger, in order to detect FFB's which are more nearly spherical than rod-like, because of de-mag field considerations applicable to more spherically-shaped FFB's. As stated above, from the torque point of view, spherical FFB's tend to be less problematic, with less risk for tissue damage than elongated FFB's, which are more likely to be rotated to align with the magnetic field, thereby causing potentially severe tissue damage. This is the “propeller” effect.

It should be emphasized that the applied magnetic field requirement of the present invention must be small enough that, in and of itself, the pre-MRI screening instrument poses minimal risk; yet, the applied magnetic field must be large enough that detectability of a hazardous FFB is achieved. Detectability depends not only upon the magnetic field applied, but also upon the size of the retained FFB, with tiny FFB's posing a greater detectability challenge. In the present invention, the MRI on-axis fringing field supplies a sufficient magnetic field amplitude to magnetize potentially hazardous FFB's for subsequent detection, while simultaneously posing extremely minimal risk of damage related to the pre-MRI screening procedure itself.

Pre-MRI screening employing the present invention can be carried out in the same room as that which houses the MRI instrument, or, alternatively, can be carried out in a separate ante-chamber.

If the pre-MRI screening is carried out in the same room which houses the MRI instrument, in the preferred embodiment, the patient and the sensing system are moved together (for instance, the patient may be placed upon a moving carriage, such as the MRI carriage itself) along the on-axis portion of the fringing magnetic field of the MRI machine. Measurements are made in serial fashion as the magnetic field increases, for example, in the range of 5 Oe to 5,000 Oe, i.e., until it is determined with a very high degree of certainty that no FFB is present in the sensed body region.

In practice, magnetic field “test zone set points” are established and then subsequently utilized for pre-MRI screening. Examples of such test zone set points are 5 Oe, 50 Oe, 75 Oe, 100 Oe, 180 Oe, and 200 Oe. Higher magnetic field strengths can alternatively be employed, such as 300 Oe, 500 Oe, 1,000 Oe, and even higher, but the higher magnetic field strengths are potentially more dangerous if, in fact, a ferromagnetic foreign body is present. For instance, a magnetic field strength of 5,000 Oe poses considerably more risk that a 200 Oe magnetic field strength. It is therefore preferred to begin with a lower magnetic field strength test zone set point, and, in serial fashion, move to a higher magnetic field strength test zone set point. One or more of these test zone set points may be utilized in the present invention's pre-MRI screening protocol, and, if desired, alternative test zone set points other than those designated above may be used. Preferentially, the test zone set points lie along the axial portion of the MRI fringing field to readily enable balancing out (nulling) unwanted magnetic field components of the MRI fringing field. Employing the axial portion of the MRI fringing field avoids the necessity of nulling asymmetrical field components of the MRI fringing field.

Knowing in advance the null setting for each designated test zone set point before the patient is introduced to the sensor system is convenient and time saving. Therefore, the null settings for the selected test zone set points can be established before the patient is positioned for pre-MRI screening, referred to herein as the step A null. As the patient's sensed body region, such as the head, has diamagnetic susceptibility which modifies the imposed applied magnetic field, however, prior to actual patient measurement, the system is re-nulled (“fine-tuned”) with the patient in position at each designated test zone set point with no motion of the sensed region, referred to herein as the step B null. Deviations from this step B null by virtue of motion of the sensed region facilitate detection of a retained FFB in the respective sensed region. For instance, eye motion provides detection capability for a FFB within the eye. Head movement provides detection capability for a FFB within the brain, or within the orbit. An absence of deviation from the final step B null, in spite of motion of the sensed region, indicates that no FFB is present. There can be an exception in the case of a previously magnetized foreign body, such as a magnet or magnet fragment, and this exception will be discussed below.

The preferred embodiment of the present invention utilizes a bimodal detection strategy using two test zone set points, as this configuration detects not only FFB's which are not previously magnetic, but also FFB's which are in and of themselves magnets. This can be useful in view of the fact that annealing or pounding upon a ferromagnetic material can result in magnetization of that material. With such a bimodal detection strategy, the likelihood of missing a FFB, whether a magnet or not, is greatly diminished. The benefit, then, of employing more than one test zone is for the rare, but yet possible, case in which the FFB is already a magnet. In such an instance, the applied magnetic field provided by the MRI fringing field at the first designated test zone set point could conceivably be just sufficient to effectively negate the magnetization of the FFB, and detection would fail, in spite of movement of the sensed region. In the next designated test zone set point, however, with a larger applied magnetic field present, detectable magnetization will now be induced, and with movement of the sensed region, a deviation from null will occur. The magnet fragment will then be detected. For instance, if detection failed at the 100 Oe test zone set point, detection would occur at a test zone set point chosen between 175 Oe and 200 Oe, such as 180 Oe. Other pairs of test zone set points can be employed, including but not limited to, 50 Oe and 100 Oe, 75 Oe and 150 Oe, or 125 Oe and 300 Oe, and so on, depending upon clinical research studies and the preference of the MRI center utilizing the present invention.

The preferred embodiment of the present invention, then, is to utilize two test zone set points. More that two test zone set points may be employed, thereby increasing the overall reliability of the system. Ultimately, the number of test zone set points shown to be most effective may be determined after large-scale clinical applications of the present invention in hospital magnetic resonance imaging departments and other free-standing MRI centers, analyses of the resultant data, and reasoned conclusions thereof, consistent with high-level scientific inquiry and medical practice.

The sensor apparatus of the present invention is best used to achieve the required measurements in a predetermined alignment relative to the MRI field axis. Preferably, the axial portion of the MRI fringing field is employed, as this facilitates the nulling process and avoids the necessity of nulling asymmetrical, off-axis magnetic field components of the MRI fringing field.

MRI systems typically provide an axially-moving carriage to support the patient. The MRI carriage, therefore, can be utilized to establish the appropriate alignment of the sensor apparatus and the patient with respect to the axial fringing field of the MRI system. The MRI carriage, then, by moving along the axial portion of the MRI fringing field, lends itself to maintaining the desired orientation for the test apparatus and the patient. In instances, therefore, in which the MRI instrument in question has a magnetic field which decays in a suitable manner such that the MRI carriage can be employed, test zone set points of varying magnitude may be utilized while the patient is lying on the MRI carriage, as the carriage is advanced along the MRI fringing field toward the MRI-measurement zone.

In some cases, the MRI carriage may be unavailable for pre-MRI screening, if it is occupied by another patient undergoing an MRI procedure. In a busy MRI center, such delays may pose an unacceptable time cost. Since an MRI magnetic field is axially symmetric, i.e., two-ended, the non-used end of the MRI machine should be vacant. This non-used end may be utilized at any time for pre-MRI screening procedures completely independently of the use of the MRI machine itself for clinical magnetic resonance imaging. In fact, by placing the second, non-used, but still magnetically active, end in close approximation to a wall, an ante-chamber for pre-MRI screening may be constructed on the far side of the wall. This configuration is not only efficient, alleviating the time-cost objection described above, but also provides excellent patient privacy.

In the single test zone set point embodiment, the sensor system is permanently placed at the desired axial magnetic fringing field location, such as that corresponding to 100 Oe. The fringing field is defined as that magnetic field of the MRI instrument outside of the measurement zone utilized for performing the magnetic resonance imaging procedure itself. Preferably, then, the sensor system is placed along the axial portion of the MRI fringing field to facilitate accurate nulling. The sensor system in this embodiment is appropriately nulled before the patient is introduced to the sensor system, in the step A null portion of the procedure. Although the step A null portion can in practice be omitted, it is recommended that the step A null be routinely performed for this single test zone set point embodiment, and for at least one test zone set point for other embodiments, because this ensures that the sensor system is functioning properly. The patient is then physically presented to the sensor system, and the sensor system is properly positioned to sense the desired body region of the patient, such as the eye. Because of diamagnetic considerations of the body region being sensed, it is necessary that the sensor system be “fine-tuned” by the step B null process. After the step B nulling, the patient is instructed to move the region being sensed, such as the eye or the head. A deviation from the step B null indicates the presence of a ferromagnetic foreign body, in which instance the MRI procedure is aborted, and the patient is taken away from the MRI magnetic field. An x-ray may then be performed to further document the presence of a retained FFB, if desired.

The single test zone set point system has the disadvantage that a foreign body which is in and of itself a magnet or magnet fragment may escape detection, if, in fact, the applied magnetic field of the MRI fringing field was just sufficient to negate the magnetization of the magnet, creating non-detectability. The solution to this problem is to employ more than one test zone set point. An efficient and effective embodiment of the method utilizes two test zone set points, and this is the preferred embodiment of the present invention.

In order to detect the magnetic field emanating from a ferromagnetic foreign body in a particular body region, such as within the eye, the orbit, or the head, it is necessary to apply a small magnetic field to that region to magnetize the FFB. The present invention, then, provides for the MRI instrument itself to function as a convenient source of applied magnetic field, which is utilized to magnetize the foreign body and thus allow it to be sensed upon movement as described herein, before subjecting the patient to the huge, and potentially dangerous, magnetic field within the MRI-measurement zone. The present invention, then, provides a practical measurement system in which the applied magnetic field is the fringing field of the MRI magnet.

In the preferred method of the present invention, the sensing apparatus system is attached, either permanently or releasably, to the carriage, or, if desired, to a suitable chair. Designated test zone set point locations, each with a corresponding null setting, are established and documented before introducing the patient to the sensor system. The null settings noted at these test-zone-set-point locations are duly recorded and used in the pre-MRI screening protocol when the step A null process is called for. It is preferred that the designated test zone set points lie along the axis of the MRI fringing magnetic field. If a carriage is employed, the patient is preferably positioned upon the carriage while in the prone position axially with respect to the MRI fringing field, as this facilitates taking measurements at the designated test zone set points.

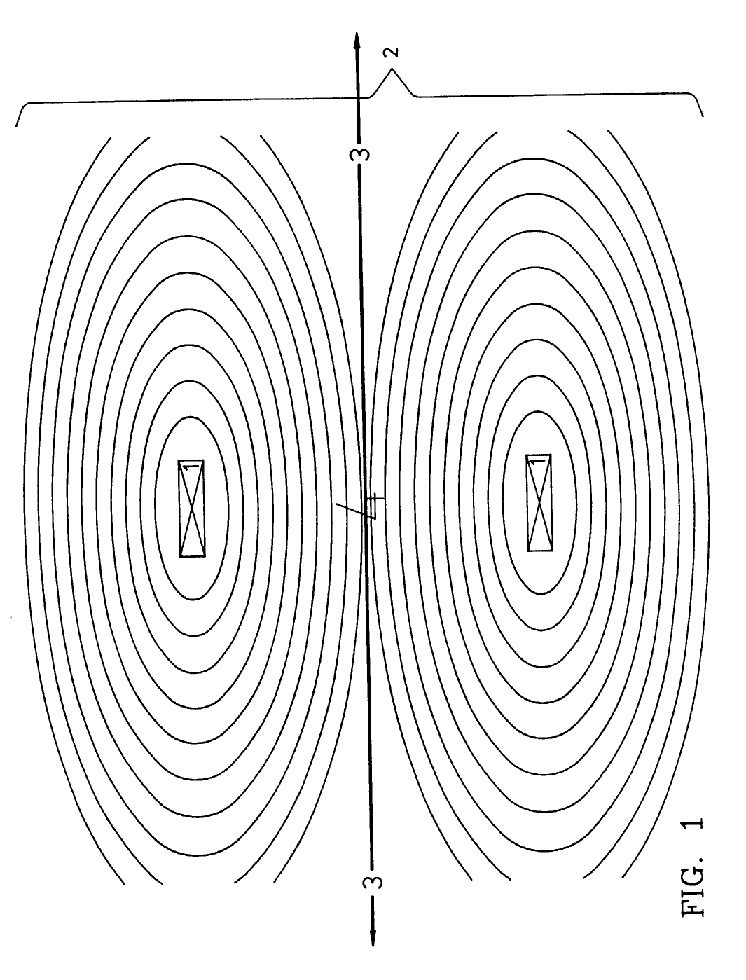

FIG. 1 depicts the magnetic field 2 of the magnets 1 of a magnetic resonance imaging (MRI) instrument. The fringing portion of the magnetic field 2 is that portion of the MRI magnetic field 2 outside of the area of the MRI-measurement zone 4. In the MRI-measurement zone 4, magnetic resonance imaging of the patient occurs. The axis 3 of the MRI fringing field passes through the MRI-measurement zone 4.

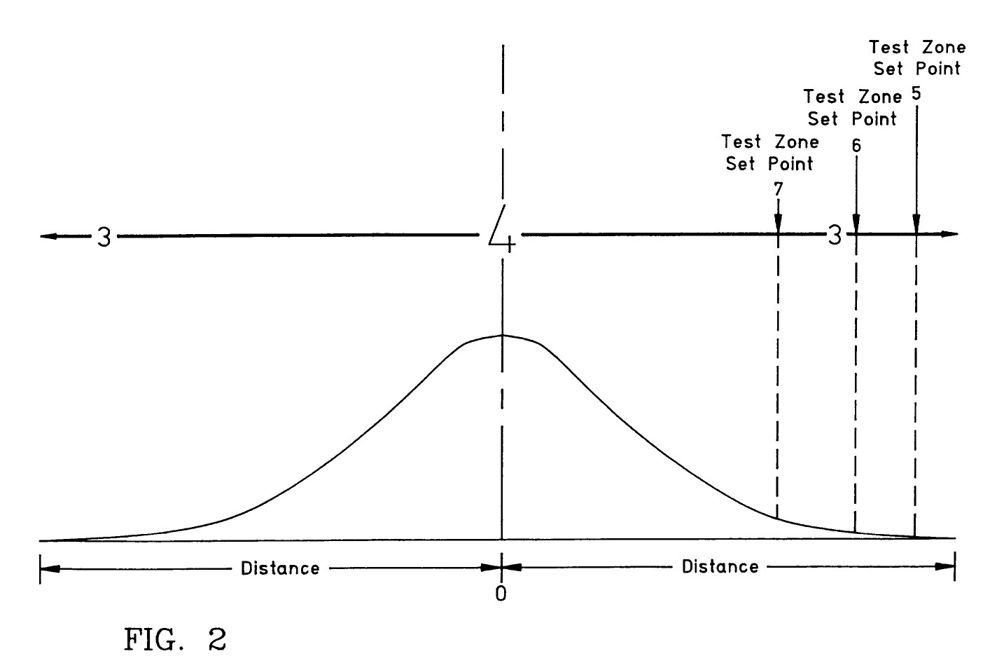

FIG. 2 depicts the diminution in magnetic field strength as the distance from the MRI-measurement zone 4 increases. Representative “test zone set points” 5,6,7 could be utilized for pre-MRI screening, as could additional points. The test zone set points 5,6,7 lie along the axial portion 3 of the MRI fringing magnetic field. The test zone set points 5,6,7, each with a corresponding null position (step A null), are established and documented. All test zone set points lie preferably along the axis 3 of the MRI fringing magnetic field to readily enable balancing out (nulling) unwanted magnetic field components of the MRI fringing field.

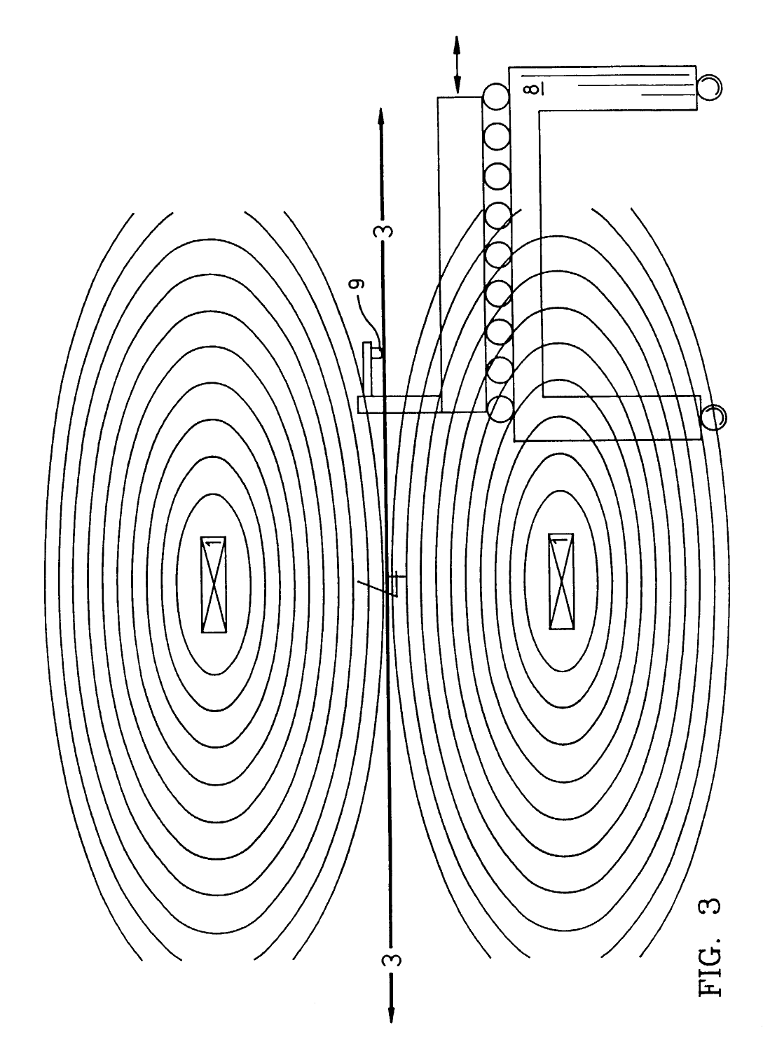

FIG. 3 shows the carriage 8, preferably the MRI carriage itself, with the attached sensor apparatus system 9. The pre-MRI screening measurements are preferentially made along the axis 3 of the MRI fringing magnetic field at a distance from the MRI magnets 1 and the MRI-measurement zone 4. The MRI fringing magnetic field supplies the applied magnetic field to magnetize a ferromagnetic foreign body, if present, and thus allow its detection by the sensor apparatus system 9.

The MRI carriage 8, therefore, can be utilized to establish the appropriate alignment of the sensor apparatus and the patient with respect to the axial portion 3 of the fringing magnetic field of the MRI system. The MRI carriage 8, by moving along the axis 3 of the MRI fringing magnetic field, lends itself to maintaining the desired orientation for the sensor apparatus system 9 and the patient.

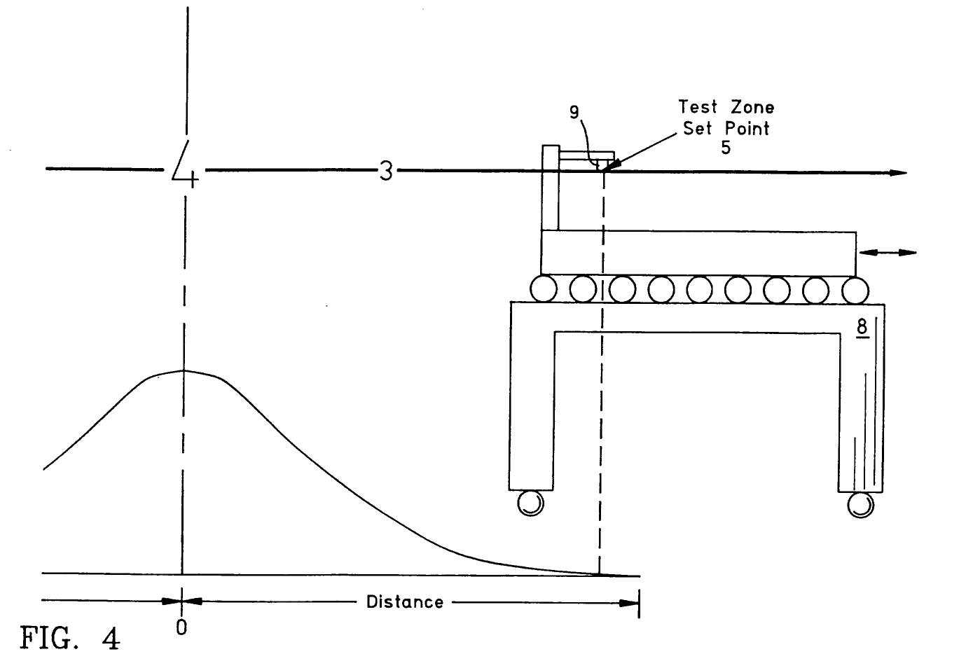

FIG. 4 depicts the MRI carriage 8 without the patient. With the sensor apparatus system 9 attached to the MRI carriage 8, the MRI carriage 8 is advanced toward the MRI instrument along the axis 3 of the MRI fringing magnetic field until the first test zone set point 5 equal to the weakest designated magnetic field strength is reached. At this point, the sensor apparatus system 9 is nulled, and the settings for step A null are documented.

In the simplest embodiment, a solitary test zone set point with its corresponding null point is utilized at the distance from the MRI magnets 1 correlating with a selected magnetic field strength, such as 100 Oe.

In a more complex embodiment, more than one test zone set point is utilized, such as, by way of example, 5 Oe, 50 Oe, 75 Oe, 100 Oe, 180 Oe, 200 Oe, 500 Oe, 1,000 Oe, and even higher, if desired. Whatever test zone set points are used, a step A null reference setting is established during the initial instrument setup for each designated test zone set point, without a patient upon the carriage.

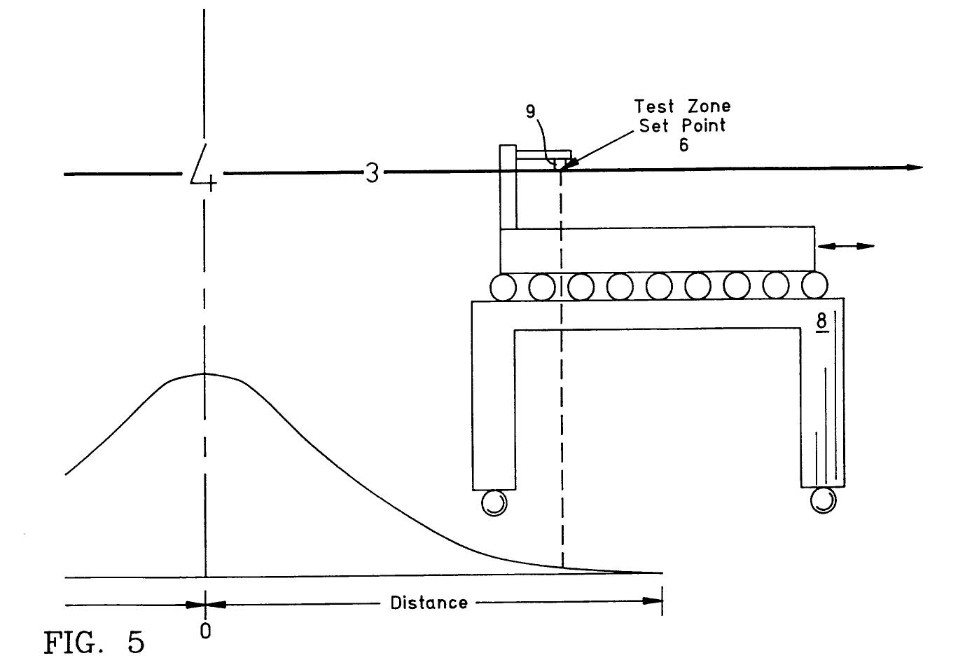

FIG. 5 shows the MRI carriage 8 advanced toward the MRI instrument along the axis 3 of the MRI fringing magnetic field until the second test zone set point 6 at the next higher designated magnetic field strength (i.e., greater magnetic field strength than the first test zone set point 5) is reached. The sensor apparatus system 9 is then appropriately nulled (step A null).

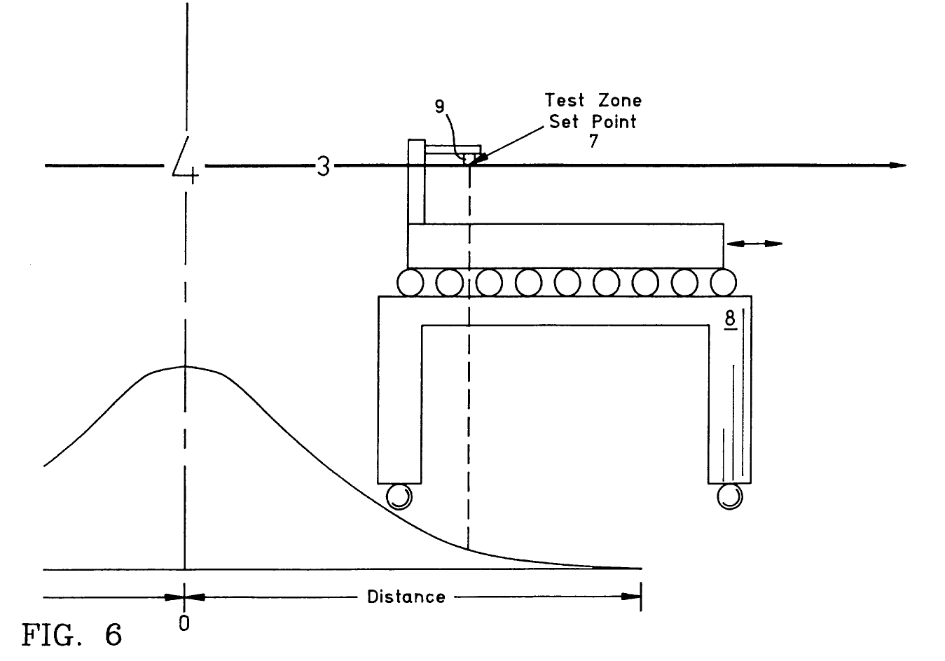

FIG. 6 shows the MRI carriage 8 advanced toward the MRI instrument along the axis 3 of the MRI fringing magnetic field until the third test zone set point 7 at the next higher designated magnetic field strength (i.e., greater magnetic field strength than the second test zone set point 6) is reached. The sensor apparatus system 9 is then appropriately nulled (step A null).

The step A null for the first test zone set point 5 with the weakest lowest magnetic field strength is performed before each patient pre-MRI screening, to make certain that the instrument is working properly. Generally, each new step A null setting for the first test zone set point will correspond with the step A null setting for this position previously established. If a significant deviation occurs, however, something may be amiss and trouble-shooting is required before confidence is regained that the instrument is operating properly.

It is generally not necessary to perform the step A null procedures at the second and third test zone set points, shown in FIGS. 5 and 6, before every patient pre-MRI screening, provided that the step A null setting at the first test zone set point 5 is similar to those previously ascertained.

In fact, it is not absolutely essential to establish step A null settings at all, and it is possible to simply go on to the step B null procedure and the patient screening. Establishing step A null settings in advance is useful for efficiency, however, and checking at least the first step A null setting without the patient on the carriage, before every patient pre-MRI screening, is advisable.

It is, therefore, very helpful, worthwhile, and recommended that step A null settings be documented when the machine is initially set up for operation in a given MRI center, and that the designated test zone set point step A null settings be confirmed on, for example, a weekly basis to insure proper instrument function. Then, using these step A null settings for reference, it becomes easier to perform the step B null (“fine-tuning”) procedure after the patient is upon the carriage. The step B null, as described herein, must be performed at every designated test zone set point, however, for every single patient. Thus, the step B null is always obligatory. Deviations from the step B null setting induced by movement of the sensed body region indicate the presence of a FFB.

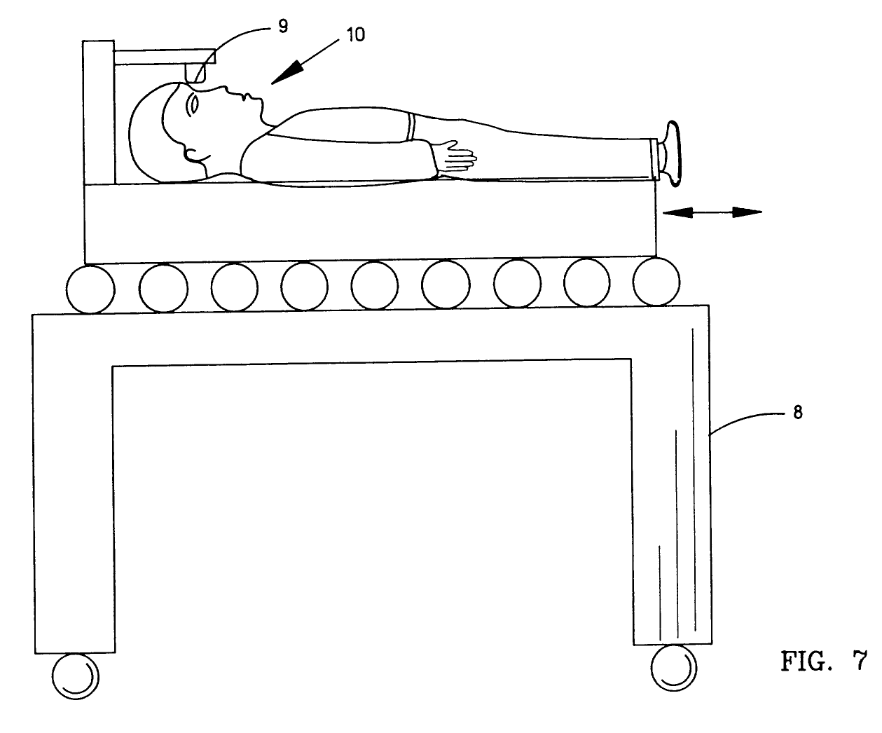

FIG. 7 shows the patient 10 placed upon the carriage 8 in the appropriate position to have the desired body region, such as the eye, sensed by the sensor apparatus system 9. The patient 10 is placed upon the carriage 8 at a distance equal to or greater from the MRI magnets than the first test zone set point 5, as depicted in FIG. 4.

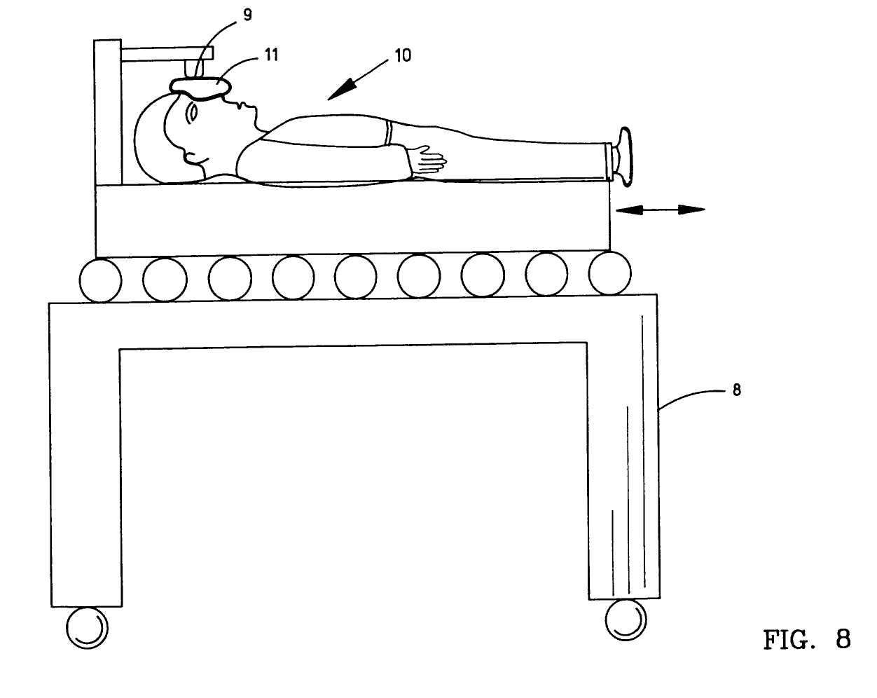

FIG. 8 shows a bag 11 of water, gel, or other appropriate material, placed between the sensor apparatus system 9 and the body region to be sensed.

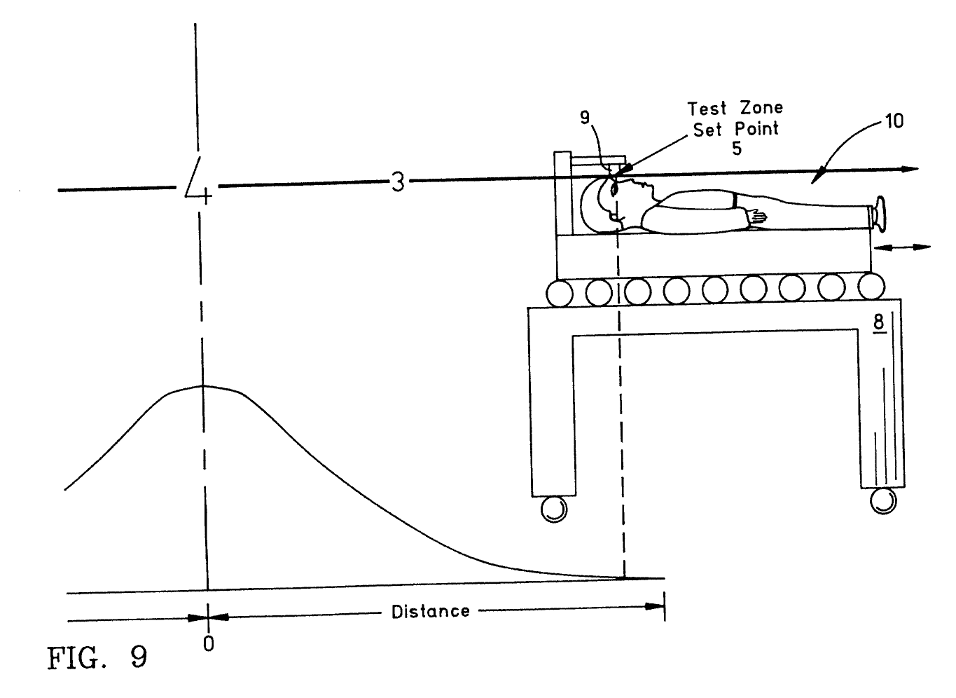

In FIG. 9, the MRI carriage 8 supporting the patient 10 is advanced along the axis 3 of the MRI fringing magnetic field to the first test zone set point 5 corresponding to the weakest selected magnetic field strength. The sensor apparatus system 9 is set according to predetermined measurements (step A null) for the first test zone set point 5. “Fine-tuning” of the null point (step B null) is performed with the sensed body region in position for FFB detection before movement of the sensed body region takes place. The patient is then instructed to move the sensed body region, and, during this movement, the sensor apparatus system 9 is interrogated. A ferromagnetic foreign body will be detected by a measurement reading deviating from the step B null. If a ferromagnetic foreign body is detected, the MRI procedure is aborted, and the patient is moved away from the MRI measurement zone 4, preferably in a slow and controlled manner to avoid dislodging the FFB, or otherwise causing it to move. If no FFB is detected at the first test zone set point 5, the MRI carriage 9 supporting the patient 10 is moved along the axis 3 of the MRI fringing field to the next designated, or second, test zone set point 6.

In the embodiment employing only a solitary test zone set point for the MRI pre-screening procedure, such as, for example, the 100 Oe test zone set point, if no FFB is detected at this test zone set point, the patient 10 can then be advanced to the MRI measurement zone 4 for the MRI procedure itself. The sensor apparatus system 9 should generally be moved out of the way, or releasably removed, before commencing magnetic resonance imaging on the patient. The potential problem with a solitary test zone set point detection system in the presence of a foreign body which in and of itself is a magnet or magnet fragment has been described.

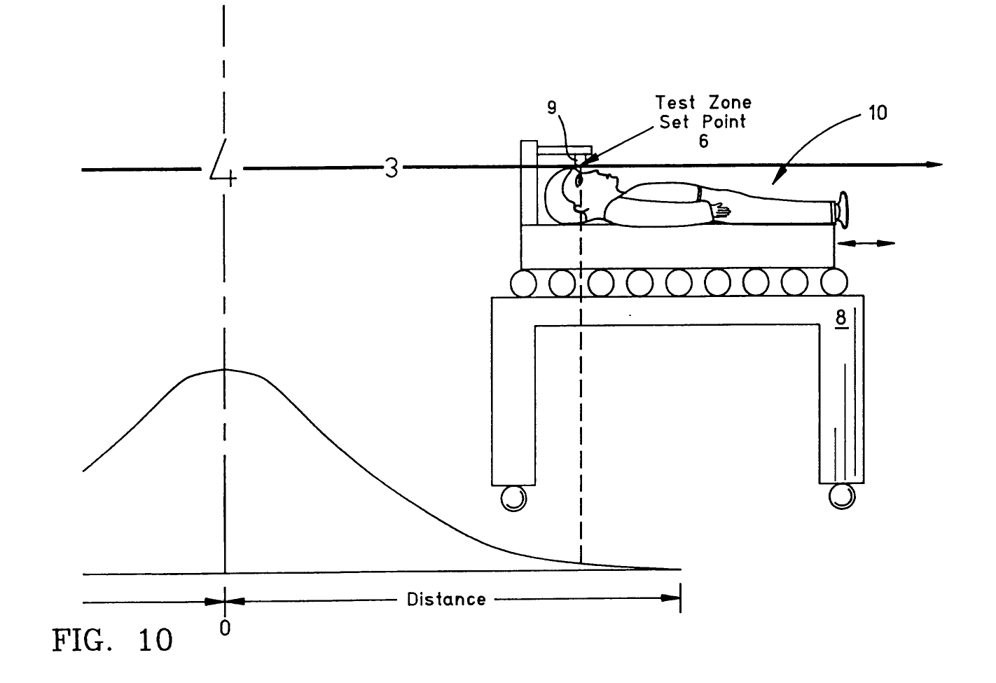

In FIG. 10, the patient 10 upon the MRI carriage 8 is advanced along the axis 3 of the MRI fringing magnetic field toward the MRI measurement zone 4 until the sensor apparatus system 9 has arrived at second test zone set point 6, which is closer to the MRI measurement zone 4 than the first test zone set point 5. The second test zone set point 6 will have a higher magnetic field strength than the first test zone set point 5. Next, the sensor apparatus system 9 is set according to predetermined measurements (i.e., those determined in the step A null) for the second test zone set point 6. “Fine-tuning” of the null point (step B null) is performed with the sensed body region in position for FFB detection before movement of the sensed body region takes place. The patient is then instructed to move the sensed body region, and, during this movement, the sensor apparatus system 9 is interrogated. A ferromagnetic foreign body will be detected by a measurement reading deviating from the step B null. If a ferromagnetic foreign body is detected, the MRI procedure is aborted, and the patient is moved away from the MRI field, preferably in a slow and controlled manner to avoid dislodging the FFB, or otherwise causing it to move. If no FFB is detected at the second test zone set point 6, the MRI carriage 8 supporting the patient 10 can be advanced along the axis 3 of the MRI fringing field to the third test zone set point 7.

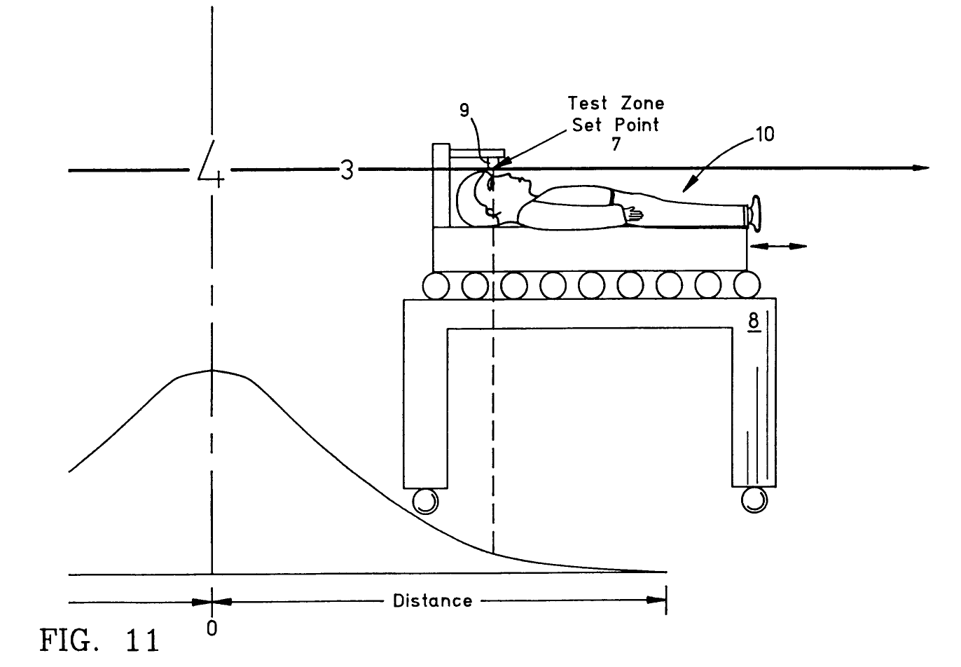

In FIG. 11, the MRI carriage 8 is advanced along the axis 3 of the MRI fringing magnetic field toward the MRI-measurement zone 4 until the sensor apparatus system 9 has arrived at the third test zone set point 7, which is closer to the MRI-measurement zone 4 than the second test zone set point 6. The third test zone set point 7 will have a higher magnetic field strength than the second test zone set point 6. The sensor apparatus system 9 is set according to predetermined measurements (step A null) for the third test zone set point 7. “Fine-tuning” of the null point (step B null) is performed with the sensed body region in position for FFB detection before movement of the sensed body region takes place. The patient is then instructed to move the sensed body region, and, during this movement, the sensor apparatus system 9 is interrogated. A ferromagnetic foreign body will be detected by a measurement reading deviating from null. If a ferromagnetic foreign body is detected, the MRI procedure is aborted, and the patient is moved away from the MRI-measurement zone 4, preferably in a slow and controlled manner to avoid dislodging the FFB, or otherwise causing it to move. If no FFB is detected at the third test zone set point 7, the MRI carriage 8 supporting the patient 10 is advanced along the axis 3 of the MRI fringing field to other selected test zone set points, as desired. The MRI procedure is not performed if a FFB is sensed at any test zone set point, and the patient is withdrawn from the MRI magnetic field. After all designated test zone set points are interrogated in the repetitive fashion described herein, if no FFB is detected, the patient is advanced upon the MRI carriage 8 into the MRI-measurement zone 4 and magnetic resonance imaging commences.

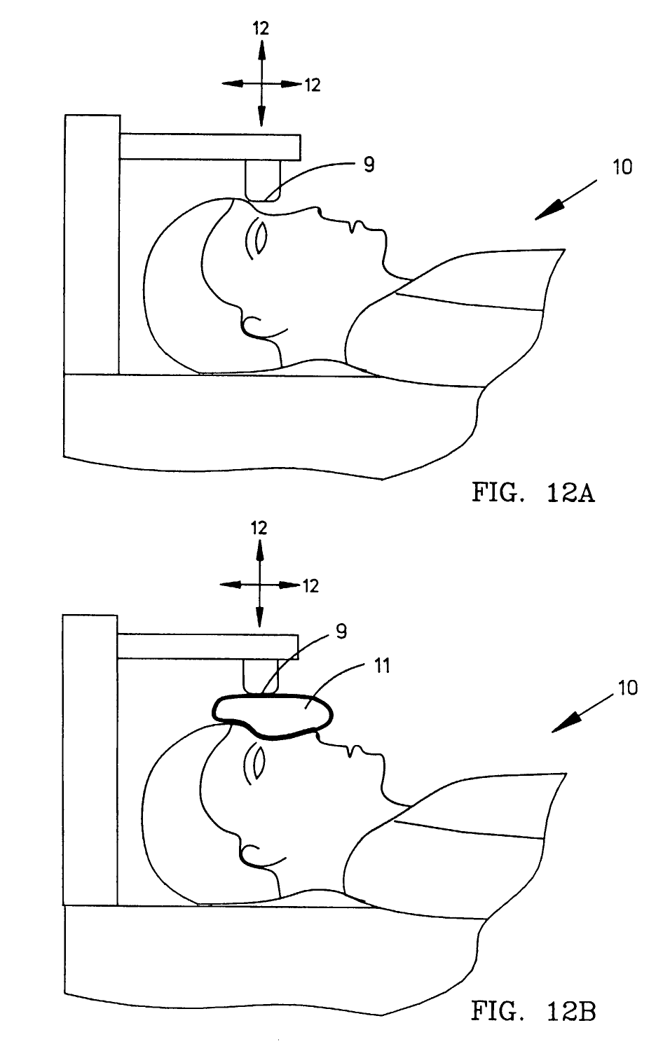

In FIGS. 12A and 12B, the patient 10 undergoes the pre-MRI screening procedure of the present invention, with the sensor apparatus system 9 sensing, if present, a FFB, upon movement of the FFB. A bag 11 of water, gel, or other appropriate material, as shown in FIG. 12B, can be used to facilitate the pre-MRI screening measurements. The patient is instructed to move the sensed body region (such as the eye, or the head) in a fashion such as shown by the arrows 12, and, during this movement, the sensor apparatus system 9 is interrogated. A ferromagnetic foreign body will be detected by a measurement deviating from the appropriate step B null setting.

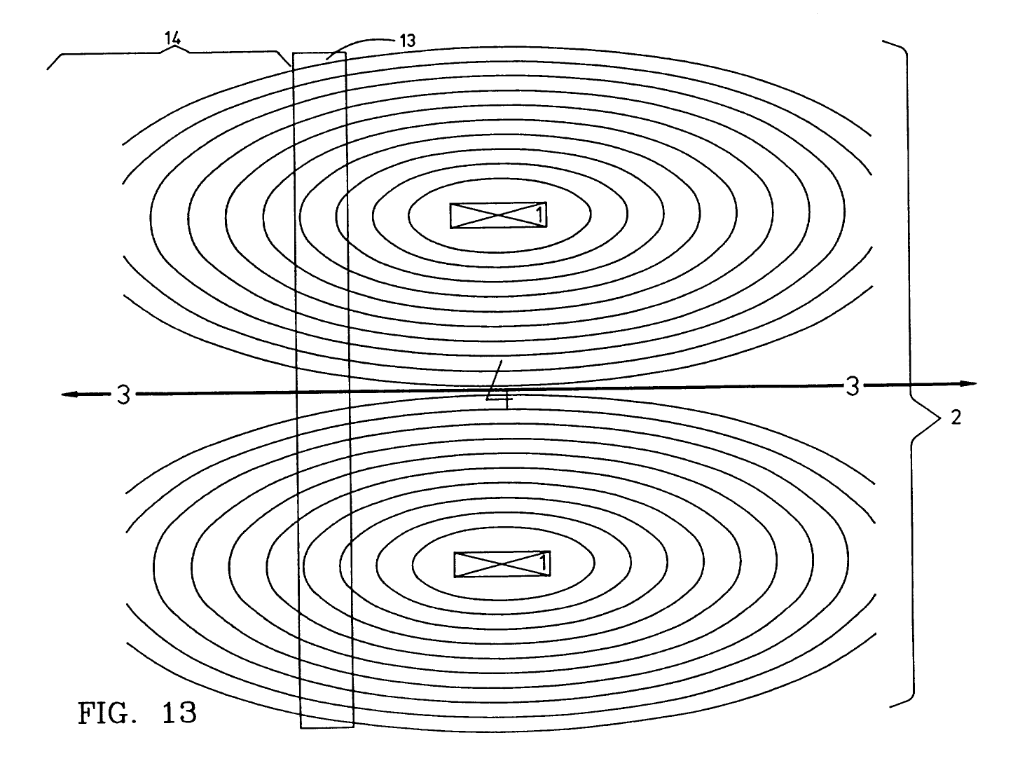

FIG. 13 depicts a way to implement the fact that the fringing magnetic field 2 of the MRI magnets 1 is axially symmetric,, i.e., it is two-ended. This fact can be successfully employed for pre-MRI screening with the present invention. The non-used area of the MRI instrument is vacant, and, therefore, may be utilized at any time for pre-MRI screening procedures, completely independent of the use of the MRI machine itself for clinical magnetic resonance imaging. In fact, by placing the second, non-used, but still magnetically active, end of the field in close relation to a wall 13, an ante-chamber 14 for pre-MRI screening may be constructed on the far side of the wall 13. This configuration is efficient, as one patient can be undergoing the MRI procedure itself within the MRI-measurement zone 4, while another patient is simultaneously being pre-screened in the ante-chamber 14 into which the MRI fringing magnetic field 2 is projected. This embodiment also provides excellent patient privacy. Pre-screening is still preferably performed along the axis 3 of the MRI fringing magnetic field 2. As noted before, the preferred embodiment utilizes more than one test zone set point, and these can be established in the ante-chamber 14.

Furthermore, it may be desirable to have the sensor system pre-positioned at the designated test zone set point within the ante-chamber corresponding to the weakest selected magnetic field strength.

Additionally, for a two test zone set point system within the ante-chamber 14, it is possible to have two independent sensor systems, with one sensor system permanently stationed at the designated test zone set point corresponding to the weaker selected magnetic field strength, and the second sensor system permanently stationed at the designated set point corresponding to the higher selected magnetic field strength. With this two-sensor pre-MRI screening system, the patient is screened sequentially by each sensor system, beginning with the weaker magnetic strength test-zone-set-point sensor system, and then proceeding with the sensor system corresponding to the higher magnetic strength test zone set point. The two test zone set points preferably lie along the MRI axis 3 of the MRI fringing magnetic field 2.

For screening of the eye, the orbit, and the head, it is possible to screen the patient in the ante-chamber in a seated position, rather than supine upon a carriage. It should be noted that it is also possible, if desired, to screen the eye, the orbit, and the head in a seated position in the same room housing the MRI instrument.

As stated herein, it is recommended and preferred that the axial portion of the MRI fringing magnetic field be used, although the scope of this invention does not preclude the use of the non-axial portion of the MRI fringing magnetic field.

The pre-MRI screening procedure can be performed in the same room housing the MRI instrument, or in a separate ante-chamber. When performing the pre-MRI screening procedure in the same room housing the MRI instrument, a typical protocol is as follows:

1. Before placing the patient on the carriage, retract the carriage (such as the MRI carriage itself) to a distance from the MRI machine equal to the weakest designated magnetic field strength test zone set point.

a. In a simplified alternative embodiment, a solitary test zone set point with its corresponding null point is utilized at the distance from the MRI magnet(s) correlating with a designated magnetic field strength, such as 100 Oe.

b. In the multiple-test-zone-set-point preferred embodiment, more than one test zone set point are utilized, such as, by way of example, 5 Oe, 50 Oe, 75 Oe, 100 Oe, 180 Oe, 200 Oe, 500 Oe, 1,000 Oe, and even higher, if desired.

c. A two-test-zone-set-point system is the preferred embodiment of the present invention, with a first test zone set point, for instance, at 100 Oe and a second test zone set point chosen between 175-200 Oe, such as at 180 Oe. Alternatively, the designated two test zone set points can be 50 Oe and 100 Oe, or 75 Oe and 150 Oe, 125 Oe and 300 Oe, or other suitable combinations of set points. By using at least two test zone set points, the likelihood of non-detection of a foreign body which is in and of itself a magnet is greatly diminished.

d. Whatever test zone set points are used, a corresponding null position, step A null, has been determined in advance for each designated test-zone-set-point location without the patient upon the carriage.

2. With no patient upon the carriage and the carriage retracted, such that the sensor system attached to the carriage is positioned along the axial MRI fringing field at the location corresponding to the weakest designated magnetic field strength, set the sensor system to the pre-established step A null position. Double-check this null to make certain that the instrument is working properly.

3. Place the patient upon the carriage, such that the sensor is appropriately positioned to measure at the weakest designated test zone set point the body region of the patient to be sensed, such as the patient's eye, or head. If desired, a bag of water, gel, or other appropriate material, may be placed between the sensor system and the body region to be sensed to facilitate the pre-MRI screening measurements and to provide increased patient comfort.

4. Now that the patient is upon the carriage and is properly position for pre-MRI screening at the first test zone set point corresponding to the weakest designated magnetic field strength, re-null the sensor apparatus, step B null, to “fine-tune” the sensor. This re-nulling is performed without movement of the body region to be sensed. Re-nulling is necessary because the body region to be inspected for an FFB, such as the head, has diamagnetic susceptibility which modifies the imposed applied magnetic field, so that it is necessary to re-establish the null before commencing movement of the body region and subsequent interrogation of the sensor apparatus.

5. The patient is then instructed to move the body region to be sensed, preferably in a controlled fashion. For eye detection, it is desirable to have a target which can be readily seen and tracked by the patient. Controlled movements enhance coherency, and thereby detectability, of the signal emanating from the FFB which has been magnetized by the MRI fringing magnetic field. The movement distinguishes the FFB from background noise.

6. The sensor apparatus is then interrogated while the sensed body area is moved.

This movement provides detectability of the FFB, and such detection will be indicated by a measurement reading deviating from the step B null.

7. If only one test zone set point is used in the MRI pre-screening procedure, such as the 100 Oe test zone set point, and no FFB is detected at this test zone set point, the patient can then be advanced into the MRI-measurement zone for the MRI procedure itself. The sensor apparatus should generally be moved out of the way, or releasably removed, before commencing magnetic resonance imaging on the patient.

8. If a ferromagnetic foreign body is detected, however, the MRI procedure is aborted, and the patient is removed from the MRI field, preferably in a slow and controlled manner to avoid dislodging the FFB, or otherwise causing it to move. All movements of the carriage should be done slowly and cautiously, to minimize risk to the patient in the unlikely event that the screening instrument somehow does not detect the presence of a ferromagnetic foreign body, or is in some way malfunctioning.

9. In the preferred embodiment utilizing multiple test zone set points, if no ferromagnetic foreign body is detected at the first test zone set point, move the carriage supporting the patient along the axial portion of the MRI fringing magnetic field toward the MRI-measurement zone until the next designated test zone set point is reached. This new test zone set point will have a greater magnetic field strength than the previous test zone set point. Generally, the patient is upon the carriage at this second test zone set point in the pre-MRI screening protocol, and it is cumbersome and unwieldy to remove the patient from the carriage at this test-zone-set-point location.

10. Next, appropriately set the sensor apparatus according to predetermined measurements for this second test zone set point by using the pre-established step A null for this location.

11. With the patient in place for appropriate FFB sensing, perform the step B null (“fine-tuning”) at this second test zone set point. Then, instruct the patient to move the body region to be sensed, preferably in a controlled fashion. Interrogate the sensor apparatus system. A ferromagnetic foreign body will be detected by a reading deviating from null induced by movement of the magnetized FFB, unless, as noted previously, the FFB is a magnet or magnet fragment. In such a rare case, if the magnet foreign body was not detected at the first test zone set point because the applied field effectively negated the magnetization of the foreign body magnet or magnet fragment, the use of a larger applied magnetic field at this second designated test zone set point will induce detectable magnetization, and, with movement of the sensed region, a measurable deviation from null will now occur.

Hence, the magnet or magnet fragment will then be detected. For instance, if detection failed at the 100 Oe test zone set point, detection should occur at a test zone set point in the range of 175-200 Oe, such as 180 Oe.

12. If a ferromagnetic foreign body is detected at this second test zone set point, the MRI procedure is aborted, and the patient is removed from the MRI field, preferably in a slow and controlled manner to avoid dislodging the FFB, or otherwise causing it to move.

13. If no ferromagnetic foreign body is detected at the second test zone set point, and, if it is desired to pre-MRI screen the patient at more than two test zone set points, move the carriage supporting the patient to the next designated test zone set point corresponding with a still greater magnetic field strength, and set the sensor apparatus according to the predetermined null setting for this third test zone set point, which was documented during the initial set-up step A null, i.e., before the patient was placed upon the carriage and introduced to the sensor system. As with the second test zone set point, it is likely cumbersome to remove the patient from the carriage, keeping in mind that confirmation that the instrument is in good working order was achieved by double-checking the first step A null, as in described above.

14. With the patient appropriately positioned upon the carriage for pre-MRI screening at this third test zone set point, the step B null proceeds. Next, instruct the patient to move the sensed body region, and interrogate the sensor system to determine the presence or absence of a FFB.

15. Repeated interrogation as described above proceeds until the designated test zone set point with the greatest magnetic field strength is interrogated, aborting the MRI if a deviation from the step B null is noted at any test zone set point. After all desired test zone set points are interrogated and noted to be negative for the detection of a ferromagnetic foreign body, the patient may then undergo magnetic resonance testing safely, with a very high degree of confidence.

The present invention is aptly suited to telemedicine, and the preferred telemedicine vehicle is the Internet. Artificial intelligence modalities, including neural net and other expert systems, can also be used, providing instantaneous autointerpretation of test results. Provision is made for providing real-time interactive feedback between the remote instrument and a central computer processing station, helping to ensure the appropriate functioning of the instrument, the use of proper test protocols, and reliable data acquisition.

The present invention has the capability, via appropriate software analyses, to determine the location, the size, and the magnetic characteristics of a retained ferromagnetic foreign body.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

We claim:

1. A method for screening a patient for the presence of a ferromagnetic foreign body, comprising:
generating a magnetic field having a first region with a strength suitable for magnetic resonance imaging and a second region remote from said first region, said second region having insufficient strength for magnetic resonance imaging;
positioning a desired portion of a patient in proximity to a magnetic sensor at a screening location within said second region of said magnetic field;
nulling the magnetic sensor output at said screening location;
orienting said desired portion of the patient at a plurality of orientations, at said screening location;
sensing magnetic susceptibility responses from said desired portion of the patient at said plurality of orientations, with said magnetic sensor; and
outputting data corresponding to said magnetic susceptibility responses.

2. The method recited in claim 1, wherein said nulling of said magnetic sensor output is performed while said desired portion of the patient is positioned at said screening location.

3. The method recited in claim 2, further comprising nulling said magnetic sensor output at said screening location when said desired portion of the patient is not positioned at said screening location.

4. The method recited in claim 1, wherein said screening location is on the axis of said magnetic field.

5. The method recited in claim 1, wherein:
said first region is located in a first room where a magnetic imaging machine is located; and
said second region is located in a second room separate from said first room.

6. The method recited in claim 1, further comprising:
positioning said desired portion of the patient in proximity to a magnetic sensor at a second screening location within said second region of said magnetic field;
nulling magnetic sensor output at said second screening location;
orienting said desired portion of the patient at a plurality of orientations at said second screening location;
sensing additional magnetic susceptibility responses from said desired portion of the patient at said plurality of orientations, at said second screening location; and
outputting data corresponding to said additional magnetic susceptibility responses.

7. The method recited in claim 6, wherein said nulling of said magnetic sensor output is performed while said desired portion of the patient is positioned at said second screening location.

8. The method recited in claim 7, further comprising nulling said magnetic sensor output at said second screening location when said desired portion of the patient is not positioned at said second screening location.

9. The method recited in claim 6, further comprising:
sensing said magnetic susceptibility responses with a first said magnetic sensor positioned at said first screening location; and
sensing said additional magnetic susceptibility responses with a second said magnetic sensor, different from said first magnetic sensor, positioned at said second screening location.

10. The method recited in claim 6, further comprising:
sensing said magnetic susceptibility responses with said magnetic sensor positioned at said first screening location; and
sensing said additional magnetic susceptibility responses with the same said magnetic sensor positioned at said second screening location.

11. The method recited in claim 6, wherein said magnetic field has a different strength at said second screening location than at said first screening location.

12. The method recited in claim 1, wherein said magnetic field has a strength at said screening location no greater than approximately one third of the strength of said first region.

13. The method recited in claim 12, wherein said strength of said magnetic field at said screening location is no greater than approximately 1000 Oe.

14. A method for screening a patient for the presence of a ferromagnetic foreign body, comprising:
generating a magnetic field having a first region with a strength suitable for magnetic resonance imaging and a second region remote from said first region, said second region having insufficient strength for magnetic resonance imaging;
sequentially positioning a desired portion of a patient at a plurality of screening locations within said second region of said magnetic field;
nulling magnetic sensor outputs at each of said plurality of screening locations;
orienting said desired portion of the patient at a plurality of orientations, at each of said screening locations;
sensing a plurality of magnetic susceptibility responses from said desired portion of the patient at said plurality of orientations, at each of said screening locations; and
outputting data corresponding to said plurality of magnetic susceptibility responses.

15. The method recited in claim 14, wherein said nulling of said magnetic sensor output at each said screening location is performed while said desired portion of the patient is positioned at each said screening location.

16. The method recited in claim 15, further comprising nulling said magnetic sensor output at each said screening location when said desired portion of the patient is not positioned at said screening location.

17. The method recited in claim 14, further comprising:
sensing said magnetic susceptibility response with a first magnetic sensor positioned at a first said screening location; and
sensing each additional said magnetic susceptibility response at each additional said screening location with a different said magnetic sensor positioned at each said additional screening location.

18. The method recited in claim 14, further comprising:
sensing said magnetic susceptibility response with a magnetic sensor positioned at a first said screening location; and
sensing said additional magnetic susceptibility responses at additional said screening locations with the same said magnetic sensor positioned at each additional said screening location.

19. The method recited in claim 14, wherein said magnetic field has a different strength at each said screening location.

20. The method recited in claim 14, wherein each said screening location is on the axis of said magnetic field.
